(19) United States
(12) Patent Application Publication
(10) Pub. No.: US 2020/0206135 A1
(43) Pub. Date: Jul. 2, 2020

(54) INSTANT THICKENED DRY FOOD COMPOSITIONS

(71) Applicant: SOLAE LLC, St. Louis, MO (US)

(72) Inventors: AMRISH CHAWLA, St. Louis, MO (US); Grace Arney, St. Louis, MO (US); Jason Mueller, St. Louis, MO (US); David M. McKeage, St. Louis, MO (US)

(21) Appl. No.: 16/735,825

(22) Filed: Jan. 7, 2020

Related U.S. Application Data
(63) Continuation of application No. 15/551,333, filed on Aug. 16, 2017, now abandoned.
(60) Provisional application No. 62/133,838, filed on Mar. 16, 2015.

(51) Int. Cl. A61K 9/127; A23L 29/269; A23L 29/212; C12N 15/88; C12N 15/113; A61K 45/06; A61K 9/00; A61P 35/00; A23C 19/09; A23L 9/10; A23L 27/60; A23L 25/10; A23L 23/10; A23L 33/00; A23L 29/262; A23L 29/256; A23L 29/231; A23L 33/185; A23L 29/25; A23L 29/238; A23L 29/20

(52) U.S. Cl. CPC A61K 9/1271; A61K 9/5068; A23L 29/212; C12N 15/88; C12N 15/1135; C12N 15/113; A61K 45/06; A61K 9/0019; A61P 35/00; A23C 19/0904; A23L 9/10; A23L 27/60; A23L 25/10; A23L 23/10; A23L 33/40; A23L 29/262; A23L 29/256; A23L 29/231; A23L 33/185; A23L 29/25; A23L 29/238; A23L 29/20; C12N 2310/14; A23L 29/269

(57) ABSTRACT

An instant thickening dry food composition. The instant thickening dry food composition includes soy protein and a mineral. The soy protein is present in an amount ranging from 3% to 99% by weight of the instant thickening dry food composition. The instant thickening dry food composition has a total hydrocolloid content of not more than 1% by weight of the instant thickening dry food composition. The instant thickening dry food composition forms an instant thickened food product when added to a liquid.

### CROSS-REFERENCE TO RELATED APPLICATIONS

This application is a Continuation of application Ser. No. 15/551,333 which represents a national filing under 35 U.S.C. 371 of International Application No. PCT/US16/22547 filed Mar. 16, 2016 and claims priority to and the benefit of U.S. Provisional Patent Application No. 62/133,838 filed Mar. 16, 2015, the contents of all prior applications are incorporated herein by reference in their entirety for all purposes.

### FIELD

The disclosure relates to instant thickening dry food compositions, instant thickened food products, and methods of making and using the instant thickening dry food compositions and instant thickened food products.

### BACKGROUND

Dysphagia is a condition in which individuals have difficulty with food mastication and swallowing. While there are a variety of causes of dysphagia and varying levels of affliction, swallowing difficulties can be dangerous, as they could result in aspiration of thin liquids and choking on solid foods. Swallowing difficulties often discourage fluid or food intake leading to inadequate hydration and nutrition in the diets of dysphagic subjects. Rheological properties of food are very important for the swallowing process. The National Dysphagia Diet (NDD) published in 2002 by the American Dietetic Association proposed rheological definitions for classifying products for subjects with different severities of dysphagia. The proposed terms for the viscosity ranges are Thin: 1-50 centiPoise (cP), Nectar-like: 51-350 cP, Honey-like: 351-1750 cP, and Spoon-thick: >1750 cP. As such, foods with textural modifications such as thickened food purees and thickened beverages are employed to facilitate “safe” swallowing, minimizing the risk of aspiration and related swallowing complications. However, currently available foods intended for consumption by dysphagia subjects have less than optimal textures and mouthfeel. For instance, liquids thickened with starches can be opaque and have a grainy and/or pulpy texture, and liquids thickened with gums have a gloppy and a slimy mouthfeel. Additionally, the inclusion of high levels of proteins to enhance the nutritional profile of the foods can produce products that have less than optimal textures and mouthfeel.

There have been several attempts to prepare dried products that can be reconstituted, i.e. so-called “instant foods”, to form acceptable food products for dysphagic subject. Satisfactory “instant foods” are meant as dry preparations which can be stored indefinitely and which can be reconstituted simply by adding water and stirring the mixture for less than five minutes to produce a product having a texture, taste and nutritional properties that are appropriate for dysphagic individuals. However, it has heretofore not been feasible to produce a satisfactory instant food having a high protein content with the desired taste, texture, and mouthfeel that are acceptable for consumption by dysphagia subjects. Thus, there is a need in the art for instant food products having an increased amount of protein and overall nutritional profile that dysphagic subjects desire and that can lead to adequate hydration and nutrition of dysphagic subjects.

Thickened products have long been known for various applications in the food industry. Typically, products are thickened by including hydrocolloids such as a gum or starch. Applicants have unexpectedly discovered that products may be thickened without including hydrocolloids such as gums or starches.

### SUMMARY

In one aspect, the disclosure provides an instant thickening dry food composition. The instant thickening dry food composition includes soy protein in an amount ranging from 3% to 99% by weight of the dry food composition. The instant thickening dry food composition further includes a mineral and a total hydrocolloid content of not more than 1% by weight of the instant thickening dry food composition. The instant thickening dry food composition forms an instant thickened food product when added to a liquid.

### BRIEF DESCRIPTION OF THE SEVERAL VIEWS OF THE DRAWINGS

FIG. 1 shows the viscosity of products produced when combining various concentrations of a mineral and soy protein (Supro® 660).

FIG. 2 shows the viscosity of products produced when combining various forms of soy protein with a mineral.

FIG. 3 shows the viscosity of products produced when combining various protein sources with a mineral.

### DETAILED DESCRIPTION OF THE PREFERRED ASPECTS

The disclosure provides instant thickening dry food compositions which include soy protein and a mineral. The compositions form instant thickened food products when added to a liquid. Non-limiting examples of food products that may be formed using instant thickening dry food compositions of the present disclosure include dessert-type instant food products such as yogurt pudding, icings, dips, frozen confections such as sherbet, mellorine, frozen yogurt, frozen custard, popsicles, sorbet, gelato, or combinations thereof, gels, soups, dips, baby foods, spreads such as peanut and cheese spreads, and the like, custards, cheese/cheese imitation, and beverages.

Advantageously, the instant thickening dry food compositions can be reconstituted to form food products having rheological properties of thickened foods recommended for dysphagic subjects in accordance with the National Dysphagia Diet (NDD) guidelines. These food products include food products having viscosities greater than 50 centiPoise (cP), including nectar-thick foodstuffs having a viscosity of 51-350 cP, honey-thick foodstuffs having a viscosity of 351-1750 cP, and spoon-thick foodstuffs having a viscosity of greater than 1750 cP. Rheological properties of thickened food products and methods of measuring rheological properties of thickened food products may be as described in the “National Dysphagia Diet; Standardization for Optimal Care” (National Dysphagia Diet Task Force (2002), ISBN-13: 978-0880913157), which is incorporated herein by reference in its entirety.

Importantly, the resultant food products have also been shown to exhibit improved nutritional characteristics, including an increased amount of protein, while retaining a taste, structure, aroma, and mouthfeel that a subject with dysphagia would desire. Methods of using the instant thickening dry food compositions to prepare instant thickened food products, and instant thickened food products made using the instant thickening food compositions are also disclosed. Various aspects of the invention are described in further detail in the following sections.

### I. Instant Thickening Compositions

Instant thickening dry food compositions of the present disclosure include soy protein and a mineral that produce a thickened food product when mixed with a liquid. The food compositions are generally prepared by mixing or blending the various ingredients using methods well known in the art. The soy protein and mineral are described in more details below.

### A. Protein Component

In general, the soy protein material may be derived from whole soybeans in accordance with methods known in the art. The whole soybeans may be standard soybeans (i.e. non-genetically modified soybeans), genetically modified soybeans (e.g., soybeans with modified oils, soybeans with modified carbohydrates, soybeans with modified protein subunits, and so forth) or combinations thereof. A variety of soy protein materials may be used in the dry food compositions of the invention. Suitable examples of soy protein material include soy extract, soymilk powder, soy curd, soy flour, soy grits, soy whey protein (2S proteins), soy protein isolate, soy protein concentrate, and mixtures thereof. Soy protein material may also be protein nutritional compositions comprising as one of the ingredients soy extract, soymilk powder, soy curd, soy flour, soy grits, soy protein isolate, soy whey protein (2S proteins), soy protein concentrate, and mixtures thereof. Soy protein material may also include hydrolyzed soy protein material.

In certain embodiments, the soy protein material may be a soy protein concentrate, which has a protein content of about 65% to less than about 90% on a moisture-free basis. Examples of suitable soy protein concentrates useful in the invention include the PROCON™ product line and the ALPHA™ product line, and identity-preserved versions of the products, all of which are commercially available from Solae, LLC. In a preferred alternative of the embodiments, the soy protein material used in the composition is ALPHA™ 12 soy protein concentrate. In another preferred alternative of the embodiments, the soy protein material used in the composition is ALPHA™ 5800 soy protein concentrate.

In certain preferred embodiments, the protein component used in the compositions is a soy protein isolate (also called isolated soy protein, or ISP). In general, soy protein isolates have a protein content of at least about 90% soy protein on a moisture-free basis. The soy protein isolate may include intact soy proteins or it may comprise hydrolyzed soy proteins. The soy protein isolate may have a high content of storage protein subunits with sedimentation coefficients such as 7S, 11S, 2S, etc. Non-limiting examples of soy protein isolates that may be used as starting material in the present invention are commercially available, for example, from Solae, LLC (St. Louis, Mo.), and among them include SUPRO® 120, SUPRO® 1500, SUPRO® 1610, SUPRO® 1611, SUPRO® 500E, SURPO® 660, SUPRO® 690, SUPRO® 760, SUPRO® 770, SUPRO® 783, SUPRO® EX 32, SUPRO® EX 33, SUPRO® EX37, SUPRO® EX38, SUPRO® EX45, SUPRO® EX 46, SUPRO® PM, and SUPRO® ST identity-preserved versions of the products, and combinations thereof.

In other preferred embodiments, the soy protein material may be a protein nutritional composition which comprises, as one of the ingredients, soy extract, soymilk powder, soy curd, soy flour, soy grits, soy protein isolate, soy whey protein (2S proteins), soy protein concentrate, and mixtures thereof. For instance, the protein nutritional composition may be a soy/dairy-based nutritional ingredient comprising a soy/dairy blend alternative to whole-milk or skim-milk powder, a soy-based nutritional ingredient enriched in vitamins and minerals, or an isolated soy protein product containing isolated soy protein and stabilized calcium phosphate. The protein nutritional composition may also be fortified with essential vitamins and minerals.

The amount of soy protein material present in the dry food compositions can and will vary depending on the desired product. The amount of soy protein material is from 3 to 99% by weight of the dry food composition including at least 75%, 70%, 65%, 60%, 55%, 50%, 45%, 40%, 35%, 30%, 25%, 20%, 15%, 10%, or at least 3% by weight of the dry food composition.

In addition to the soy protein material, other optional proteins may also be present in the dry food composition. For instance, the other proteins may be derived from a plant other than soy. By way of non-limiting example, suitable plants include amaranth, arrowroot, barley, buckwheat, canola, cassava, channa (garbanzo), legumes, lentils, lupins, maize, millet, oat, pea, potato, rice, rye, sorghum, sunflower, tapioca, triticale, wheat, and mixtures thereof. Especially preferred plant proteins include barley, canola, lupin, maize, oat, pea, potato, rice, wheat, and combinations thereof.

In another embodiment, the optional protein may be derived from the muscles, organs, connective tissues, or skeletons of land-based or aquatic animals. As an example, the animal protein may be gelatin, which is produced by partial hydrolysis of collagen extracted from the bones, connective tissues, organs, etc., from cattle or other animals.

Optional protein material may also be derived from an animal source without departing from the scope of the invention. In one embodiment, the animal protein material may be derived from eggs. Non-limiting examples of suitable egg proteins include powdered egg, dried egg solids, dried egg white protein, liquid egg white protein, egg white protein powder, isolated ovalbumin protein, and combinations thereof. Egg proteins may be derived from the eggs of chicken, duck, goose, quail, or other birds. In a preferred alternate embodiment, the protein material may be derived from a dairy source. Suitable dairy proteins include non-fat dry milk powder, milk protein isolate, milk protein concentrate, acid casein, caseinate (e.g., sodium caseinate, calcium caseinate, etc.), whey protein isolate, whey protein concentrate, and combinations thereof. The milk protein material may be derived from cows, goats, sheep, donkeys, camels, camelids, yaks, water buffalo, etc.

The amount of optional protein that may be present in the instant thickening dry food composition can and will vary depending on the intended food product and the additional protein to be used, and may be determined experimentally. For instance, the amount of optional protein in an instant thickening dry composition may range from about 0% to about 30% by weight of the instant thickening dry food composition. In some embodiments, the amount of optional protein present in the instant thickening dry food composition may range from about 1% to about 20% by weight of the instant thickening dry food composition. In other embodiments, the amount of optional protein that may be present in the instant thickening dry food composition may range from about 2% to about 10% by weight of the instant thickening dry food composition. In certain embodiments, no optional protein is included in the instant thickening dry food composition.

### B. Mineral

The instant thickening dry food compositions include a mineral. In certain embodiments, the compositions include metal cations as a part of the mineral. The metal cation is preferably a metal cation with a valency of at least 2. Non-limiting examples of suitable metal cations with a valency of at least 2 include magnesium and calcium. Without being bound by theory, upon addition of a liquid to dry compositions comprising the soy protein and mineral, the composition may dissociate to increase the pH and promote solubility of the soy protein which may help unfold the protein structure allowing the proteins to form a three dimensional space-filling network and thickening the resulting food product. Additionally, the oxidative properties of the compound used may promote protein-protein interaction in the form of disulfide bonding and other intermolecular bonding. The divalent cation species may also show crosslinking with the protein to promote thickening of the food product.

In addition to the mineral, the dry food composition may include hydrocolloids. Non-limiting examples of suitable hydrocolloids that may be used in the dry food compositions of the present invention include carrageenan, cellulose gum, cellulose gel, starch, gum arabic, xanthan gum, a carbohydrate, and any other hydrocolloid known and used in the industry, and combinations thereof. As will be appreciated by a skilled artisan, the amount of hydrocolloids added to the food composition can and will depend upon the type of food product desired. These hydrocolloids will represent a small amount, i.e., the total hydrocolloids content will be not more than 1% by weight of the instant thickening dry food composition. In certain embodiments, the total hydrocolloid content is even lower such as less than 0.75%, less than 0.5%, or less than 0.25% by weight of the dry food composition. In certain embodiments, the dry food composition does not include any of these hydrocolloids.

In some embodiments, the compositions comprise a carbohydrate. Generally, a suitable carbohydrate may be sugar, maltodextrin, polydextrose, or cereal flour. Non-limiting examples of suitable sugars may include sucrose, dextrose, lactose, and fructose.

In certain embodiments, the compositions comprise an instant starch as a thickening agent. An appropriate instant starch may provide proper viscosity and mouth-feel of the composition when it has been reconstituted with a liquid. Suitable instant starches must be quick-setting, be re-hydrated rapidly, and have a bland flavor, and can be quickly reconstituted with liquid by minimal mixing by shaking or stirring. In the experience of these inventors, the preferred instant starch is an instant starch, also known as a cold water swelling (CWS) starch. Instant starch is a term used to describe granular instant starches that swell in cold systems to develop viscosity. The instant starch is typically extracted from cereals selected from the group consisting of corn, wheat, or rice, or from roots or tubers selected from the group consisting of tapioca and potato, or mixtures thereof. Examples of suitable instant starches include MIRA THIK® 470 starch manufactured by A.E. Staley Co.; H-50 Modified Starch manufactured by Ingredion; and a variety of C* HIForm brand modified starches manufactured by Cerestar/Cargill Company; CLEARAM and other modified starches manufactured by Roquette corp. As described above, where starch is present, the starch, in combination with other hydrocolloids, will represent not more than 1% by weight of the instant thickening dry food composition.

Regardless of the specific carbohydrate source used, the percentage of carbohydrate utilized in the food product typically determines, in part, its texture when it is hydrated or expanded. As such, the amount of carbohydrates present in the food product can and will vary depending on the desired texture of the food product.

In certain embodiments, the compositions comprise a stabilizer. Non-limiting examples of suitable stabilizers that may be used include pectin, agar, food gums (such as locust bean gum, xanthan gum, cellulose gum, gum arabic and guar gum), alginic acid, locust bean gum, carrageenan, gelatin, and any other stabilizer known and used in the industry. As will be appreciated by a skilled artisan, the amount of stabilizer, if any, added to the food composition can and will depend upon the type of food product desired. As described above, where a stabilizer is present, the stabilizer, in combination with other hydrocolloids, will represent not more than 1% by weight of the instant thickening dry food composition.

### C. Additional Ingredients

In addition to the ingredients detailed in A-B above, a variety of other ingredients may be added to the instant compositions without departing from the scope of the invention. For example, dietary fiber, antioxidants, antimicrobial agents, vegetable oils, animal derived fats, emulsifiers, flavoring agents, coloring agents, sequestering agents, pH-adjusting agents, preservatives, dairy products, other nutrients, probiotics, prebiotics, dry food pieces, dehydrated vegetables, dehydrated meats, dehydrated fruit or vegetable powders, and combinations thereof may be included.

The instant thickening dry food compositions may comprise a sufficient amount of at least one sweetener. Either natural and/or synthetic sweeteners can be utilized. By way of non-limiting example, the sweetener may be selected from glucose (corn syrup, corn syrup solids), dextrose, maltodextrin, invert sugar, fructose, lactose, soy molasses, monk fruit extract, maple syrup, honey, and mixtures thereof or any of the high intensity sweeteners including saccharin and its various salts such as the sodium salt, dipeptide sweeteners such as aspartame and neotame, dihydrochalcone compounds, glycyrrhizin, Stevia rebaudiana (Stevioside), chloro derivatives of sucrose such as sucralose, sugar alcohols such as sorbitol, mannitol, sylitol, and the like. Also contemplated are hydrogenated starch hydrolysates and the synthetic sweetener 3,6-dihydro-6-methyl-1,2,3-oxathiazin-4-one-2,2-dioxide, particularly the potassium salt (acesulfame-K), and sodium and calcium salts thereof.

Typically, the amount of sweetener can and will vary widely depending on the sweetener used in the compositions and the intended food product. However, generally, from about 0.01% to about 75% by weight sweetener and preferably from about 5% to 25% by weight sweetener is incorporated into the dry food composition when the sweetener is not a high intensity sweetener. When the sweetener is a high intensity sweetener, about 0.01% to about 5% by weight sweetener and preferably from about 0.01% to 1% by weight sweetener is incorporated into the dry food composition.

The instant thickening dry food compositions may comprise spray-dried fat powders. Non-limiting examples of suitable spray dried fat powders may be derived from include dairy creamers, palm oil, rapeseed oil, soybean oil, sunflower oil, canola oil, corn oil, coconut oil, and lecithin. The percent of the dry food composition comprised of spray dried fat powders will depend, in part, on the fat powder used and the desired product. For instance, spray dried fat may comprise between about 5% and 50% by weight of the dry food composition.

The dry food compositions may comprise an emulsifier. Non-limiting examples of suitable emulsifiers include distilled mono and di-glycerides, propylene glycol monoesters, sodium stearoyl-2-lactylate, polsorbate 60, lecithin, hydroxylated lecithin and any other emulsifier known and used in the industry. The percent of the pre-blend comprised of an emulsifier will depend, in part, on the emulsifier used and desired product. Generally, emulsifiers that may be combined with compositions of the present invention are generally used at low concentrations known to those skilled in the art. For instance, an emulsifier may comprise between about 0.01% and 3% by weight of the dry food composition. Preferably, an emulsifier may comprise between about 0.05% and 2% by weight of the dry food composition. More preferably, an emulsifier may comprise between about 0.5% to 1% by weight of the dry food composition.

Antioxidant additives include ascorbic acid, butylated hydroxyanisole (BHA), butylated hydroxytoluene (BHT), tert-butylhydroquinone (TBHQ), vitamins A, C, and E and associated derivatives, and various plant extracts, such as those containing carotenoids, tocopherols, or flavonoids having antioxidant properties, may also be included to increase the shelf-life or nutritionally-enhance the food product. The antioxidants may have a presence at levels generally known to those skilled in the art. Generally, the antioxidants may have a presence at levels from about 0.01% to about 1%, preferably from about 0.05% to about 3%, and more preferably from about 0.1% to about 2% by weight of the dry food compositions.

In some embodiments, it may be desirable to lower or raise the pH of the food composition depending on the type of food end product desired. Thus, the food composition may be contacted with a pH-adjusting agent. The pH of the food composition may range from about 4.5 to about 11.5. Several pH-adjusting agents are suitable for use in the invention. The pH-adjusting agent may be organic, or alternatively, it may be inorganic. In exemplary embodiments, the pH-adjusting agent is a food grade edible acid. Non-limiting acids suitable for use in the invention include lactic, citric, tartaric, malic, and combinations thereof. In an exemplary embodiment, the pH-adjusting agent is citric acid. In alternative embodiments, the pH-adjusting agent is a food grade edible base, such as but not limited to, disodium phosphate and potassium hydroxide. As will be appreciated by a skilled artisan, the amount of pH-adjusting agent contacted with the food composition can and will vary depending on several parameters, including, the agent selected and the desired pH.

The food product may optionally include a variety of flavorings, spices, or other ingredients to naturally enhance the taste of the final food product. As will be appreciated by a skilled artisan, the selection of ingredients added to the food composition can and will depend upon the type of food product desired.

In one embodiment, the food composition may further comprise a flavoring agent. The flavoring agent may include any suitable edible flavoring agent known in the art including, but not limited to, salt, any flower flavor, any spice flavor, vanilla, any fruit flavor, caramel, nut flavor, beef, poultry (e.g. chicken or turkey), pork or seafood flavors, dairy flavors such as butter and cheese, any vegetable flavor and combinations thereof. Additionally, other sweet flavors may be used (e.g., chocolate, chocolate mint, caramel, toffee, butterscotch, mint, and peppermint flavorings). A wide variety of fruit or citrus flavors may also be used. Non-limiting examples of fruit or citrus flavors include strawberry, banana, pineapple, coconut, cherry, orange, and lemon flavors.

A wide variety of spice flavors may also be used. Non-limiting examples include herb and garlic, sour cream and onion, honey mustard, hot mustard, dry roast, barbecue, jalapeno, red pepper, garlic, chili, sweet and sour seasoning, sweet seasoning, hot and spicy seasoning, savory flavor seasoning, vegetable seasonings, and combinations thereof.

The food product may optionally include an ingredient that is a dairy product. Suitable non-limiting examples of dairy products that may additionally be added to the food composition are whole milk powder, dairy-derived fat powder (e.g., creamers), cultured yogurt powder, buttermilk powder, non-fat dry milk powder, milk proteins, acid casein, caseinate (e.g., sodium caseinate, calcium caseinate, etc.), whey protein concentrate, whey protein isolate, milk protein isolate, milk protein concentrate, or any other kind of dairy powder, and combinations thereof.

In an additional embodiment, the food composition may further comprise a coloring agent. The coloring agent may be any suitable food coloring, additive, dye or lake known to those skilled in the art. Suitable food colorants may include, but are not limited to, for example, Food, Drug and Cosmetic (FD&C) Blue No. 1, FD&C Blue No. 2, FD&C Green No. 3, FD&C Red No. 3, FD&C Red No. 40, FD&C Yellow No. 5, FD&C Yellow No. 6, Orange B, Citrus Red No. 2 and combinations thereof. Other coloring agents may include annatto extract, b-apo-8′-carotenal, beta-carotene, beet powder, canthanxantin, caramel color, carrot oil, cochineal extract, cottonseed flour, ferrous gluconate, fruit juice, grape color extract, paprika, riboflavin, saffron, titanium dioxide, turmeric, and vegetable juice. These coloring agents may be combined or mixed as is common to those skilled in the art to produce a final coloring agent.

In a further embodiment, the food composition may further comprise additional nutrients such as vitamins, minerals, antioxidants, omega-3 fatty acids, and herbs. Suitable vitamins include Vitamins A, C and E, which are also antioxidants, and Vitamins B and D. Examples of minerals that may be added include the salts of ammonium, calcium, magnesium, selenium, cadmium, molybdenum, manganese, zinc, iron, iodine, and potassium. Suitable omega-3 fatty acids include docosahexanenoic acid (DHA). Herbs that may be added include basil, celery leaves, chervil, chives, cilantro, parsley, oregano, tarragon, and thyme.

In another embodiment, the food composition may further comprise a probiotic microorganism. As used herein, probiotic microorganisms (hereinafter “probiotics”) are food-grade microorganisms (alive, including semi-viable or weakened, and/or non-replicating), metabolites, microbial cell preparations or components of microbial cells that could confer health benefits on the host when administered in adequate amounts, more specifically, that beneficially affect a host by improving its intestinal microbial balance, leading to effects on the health or well-being of the host. See, Salminen S, Ouwehand A. Benno Y. et al., “Probiotics: how should they be defined?,” Trends Food Sci. Technol., 1999:10, 107-10. In general, it is believed that these micro-organisms inhibit or influence the growth and/or metabolism of pathogenic bacteria in the intestinal tract. The probiotics may also activate the immune function of the host. Non-limiting examples of probiotics include organisms belonging to the genera Aerococcus, Aspergillus, Bacillus, Bacteroides, Bifidobacterium, Candida, Clostridium, Debaromyces, Enterococcus, Fusobacterium, Lactobacillus, Lactococcus, Leuconostoc, Melissococcus, Micrococcus, Mucor, Oenococcus, Pediococcus, Penicillium, Peptostrepococcus, Pichia, Propionibacterium, Pseudocatenulatum, Rhizopus, Saccharomyces, Staphylococcus, Streptococcus, Torulopsis, Weissella, or combinations thereof.

In some embodiments, the probiotics are yogurt producing bacteria. Suitable probiotic yogurt producing bacteria may include Lactobacillus bulgaricus, Streptococcus thermophilus, and Lactobacillus acidophilus as well as other yogurt cultures and mixtures comprising yogurt producing bacteria. These bacteria are available as dry cultures and can be either used in a spray-dried, freeze-dried, or encapsulated form.

The food composition may further comprise a dietary fiber. Dietary fiber or roughage is the indigestible portion of food derived from plants. Dietary fiber generally comprises soluble fiber and insoluble fiber. Soluble fiber dissolves in water, is readily fermented in the colon into gases and physiologically active byproducts, and can have prebiotic properties. Insoluble fiber does not dissolve in water, is metabolically inert and provides bulking, or it can be prebiotic and metabolically ferment in the large intestine. As used herein, a “prebiotic” is a term used to describe a food substance, normally a dietary fiber, that selectively promotes the growth of beneficial bacteria or inhibits the growth or mucosal adhesion of pathogenic bacteria in the intestines. Prebiotics are, for example, defined by Glenn R. Gibson and Marcel B. Roberfroid, Dietary Modulation of the Human Colonic Microbiota: Introducing the Concept of Prebiotics, J. Nutr. 1995 125: 1401-1412. In some embodiments, a suitable dietary fiber is a dietary fiber with prebiotic properties. Non-limiting examples of dietary fiber with prebiotic properties include acacia gum, alpha glucan, arabinogalactans, beta glucan, dextrans, fructooligosaccharides, fucosyllactose, galactooligosaccharides, galactomannans, gentiooligosaccharides, glucooligo-saccharides, guar gum, inulin, psyllium, isomaltooligosaccharides, lactoneo-tetraose, lactosucrose, lactulose, levan, maltodextrins, milk oligosaccharides, partially hydrolyzed guar gum, pecticoligosaccharides, resistant starches, retrograded starch, sialooligosaccharides, sialyllactose, soyoligosaccharides, sugar alcohols, xylooligosaccharides, or their hydrolysates, or combinations thereof. Examples of dietary fiber with prebiotic properties that may be utilized in the invention include, for example Litesse® Ultra Polydextrose, FIBRIM® 2000, and FIBRIM® 1020 available from Danisco A/S (Copenhagen, Denmark).

In some embodiments, the compositions further comprise dry food pieces. As used herein, the term “dry food pieces” may be used to describe any dry food product having a crunchy or chewy texture. The food pieces can provide a contrasting crunchy or chewy texture upon reconstitution. Additionally, the dry food pieces may facilitate reconstitution of the food product during preparation by helping disperse the powder in the liquid. Non-limiting examples of dried food products include granola nuggets, crisp rice, flaked and/or puffed cereal, cookie bits, candies, dehydrated meats, dry vegetable or non-vegetable pieces, pelletized protein nuggets, including soy protein nuggets, pieces of nuts and dried fruits, or any other dried food pieces.

### II. Method of Preparing a Food Product Using the Dry Composition

In some aspects, the disclosure provides a method of preparing an instant thickened food product, such as a food product suitable for administration to subjects with dysphagia, using a dry food composition described in Section I above. The method comprises, in a first step, adding an instant thickening dry food composition disclosed herein to a liquid. The amount of instant thickening dry food composition added to the liquid will vary depending on the desired end product. In certain embodiments, the amount of the instant thickening dry food composition added to the liquid is an amount sufficient to produce an instant thickened food product having a viscosity of greater than 50 centiPoise. In some embodiments, the liquid is water, milk, fruit or vegetable juices, coffee, or tea. In some embodiments the liquid temperature is warm; in other embodiments the liquid temperature is ambient or colder. In a second step, the liquid to which the dry food composition is added is stirred or shaken vigorously to hydrate the dry food composition, whereby the instant thickened food product is prepared. The instant thickened food product preferably contains a sufficient amount of the instant thickening dry food composition such that the instant thickened food product has a viscosity of at least 50 centiPoise. In certain embodiments, the instant thickened food product contains at least 8%, at least 10%, at least 15%, or greater amount of the instant thickening dry food composition by weight of the instant thickened food product. Optionally, the instant thickened food product is allowed to stand after shaking or mixing to allow the dry ingredients to hydrate completely and for the texture and flavor of the resultant food product to develop.

In certain embodiments, the liquid temperature is cold. In other embodiments, the liquid temperature is hot. The liquid and the dry food composition are stirred or shaken vigorously for a sufficient amount of time to hydrate the dry food composition. In certain embodiments, sufficient hydration is obtained when a food product having a viscosity of at least 50 centiPoise, including at least 351 centiPoise, at least 1751 centiPoise, or greater viscosity is obtained. A sufficient amount of time to hydrate the dry food composition may be from about 1 second to about ten minutes or longer. Preferably, the liquid and the dry food composition are stirred or shaken vigorously for about 5, 10, 15, 20, 30, 40, or about 50 seconds or more. In preferred embodiments, the liquid and the dry food composition are stirred or shaken for about 20, 25, 30, 35, or about 40 seconds.

### III. Method of Treating a Dysphagic Subject

In other aspects, the disclosure provides a method of treating subjects with dysphagia. The method comprises preparing an instant thickened food product suitable for administration to a subject with dysphagia using an instant thickening dry food composition described in Section I above, and administering the food product to the subject with dysphagia.

The term “subject,” as used herein, refers to an animal. Suitable animals include vertebrates such as mammals, birds, reptiles, amphibians, and fish. Examples of suitable mammals include, without limit, rodents, companion animals, livestock, and primates. Non-limiting examples of rodents include mice, rats, hamsters, gerbils, and guinea pigs. Suitable companion animals include, but are not limited to, cats, dogs, rabbits, hedgehogs, and ferrets. Non-limiting examples of livestock include horses, goats, sheep, swine, cattle, llamas, and alpacas. Suitable primates include, but are not limited to, humans, capuchin monkeys, chimpanzees, lemurs, macaques, marmosets, tamarins, spider monkeys, squirrel monkeys, and vervet monkeys. Non-limiting examples of birds include chickens, turkeys, ducks, and geese. An exemplary subject is a human.

### Definitions

To facilitate understanding of the invention, several terms are defined below.

The term “degree of hydrolysis” refers to the percentage of the total peptide bonds that are cleaved.

When introducing elements of the present invention or the preferred embodiments(s) thereof, the articles “a,” “an,” “the” and “said” are intended to mean that there are one or more of the elements. The terms “comprising,” “including” and “having” are intended to be inclusive and mean that there may be additional elements other than the listed elements.

As various changes could be made in the above compounds, products and methods without departing from the scope of the invention, it is intended that all matter contained in the above description and in the examples given below, shall be interpreted as illustrative and not in a limiting sense.

### EXAMPLES

### Example 1: Preparation of a Gel with SUPRO® Isolated Soy Protein

The following example relates to a method for preparing an instant thickened food product that forms a gel that can meet the requirements for nectar-, honey-, and spoon-thickgel consistency for people with dysphagia (swallowing difficulties). The instant thickening dry food composition contains an amount of plant protein, sugar and flavor.

The instant thickened gel was formed according to typical industry processing techniques using the step-by-step process below. Table 1 is the list of ingredients used in percentage (%) by weight in grams.

TABLE 1
Instant Thickening Protein Gel Powder

| Ingredient | % Usage Range |
|---|---|
| Sugar | 40 to 60 |
| Soy protein | 30 to 50 |
| Cocoa | 2 to 8 |
| Natural Flavor | 3 to 8 |
| Mineral | 0.5 to 1.5 |
| Vitamin Premix | 0.25 to 1 |

The ingredients were combined and processed according to the following steps to produce the finished product.

All dry ingredients were mixed in a Hobart N-50 mixer using a paddle attachment on speed setting #1 for 5 to 10 minutes. The instant thickening dry food composition was then packaged into individual single serve packets containing 40 g to 60 g powder.

For the preparation of the instant thickened food product, 100 mL to 150 mL of cold water was added to an 8 oz disposable Solo™ paper cup. One single serve packet was added to the cold water and the lid was placed onto the container. The cup was then shaken vigorously by hand for 20 to 30 seconds allowing the instant thickening dry food composition to hydrate and thicken (set).

The result was an instant thickened food product containing plant protein and sugar with the taste, structure, aroma, and mouth feel of commercially-prepared gels. The addition of SUPRO® Isolated Soy Protein contributed to the improved mouth feel and texture of the finished product.

### Example 2: Preparation of a Ready to Eat Dip with SUPRO® Isolated Soy Protein and Fiber

The following example relates to a method for preparing an instant thickening powdered dip that contains an amount of plant protein, dairy based fat and fiber with the taste, structure, aroma, and mouth feel of commercially refrigerated dips.

The instant thickening dip was formed according to typical industry processing techniques using the step-by-step process below. Table 2 is the list of ingredient ranges in percentage (%) by weight in grams.

TABLE 2
Instant Thickening Dip Powder

| Ingredient | % Usage Range |
|---|---|
| Soy protein | 40 to 60 |
| Fiber | 20 to 30 |
| Seasoning mix | 8 to 15 |
| Dairy fat powder | 10 to 20 |
| Salt | 0.5 to 1.5 |
| Mineral | 0.5 to 1 |
| Natural flavor | 0.5 to 1.5 |

The ingredients were combined and processed according to the following steps to produce the instant thickened dip.

All dry ingredients were mixed in a Hobart N-50 mixer using a paddle attachment on speed setting #1 for 5 to 10 minutes. The powder was then packaged into individual single serve packets containing 40 g to 60 g.

For the preparation of the instant thickened dip, 140 mL to 160 mL of cold water was added to an 8 oz disposable Solo™ paper soup bowl. One single serve packet was added to the cold water and the lid was placed onto the container. The bowl was then shaken vigorously by hand for 20 to 30 seconds allowing the powder to hydrate and thicken (set).

The result was an instant thickened dip containing a combination of plant protein, dairy fat and fiber with the taste, structure, aroma, and mouth feel of commercially refrigerated dips. The addition of SUPRO® Isolated Soy Protein contributed to the improved mouth feel and texture of the finished product.

### Example 3: Preparation of a Protein-Fortified Pudding Type Dessert with SUPRO® Isolated Soy Protein and Dehydrated Fruit Pieces

The following example relates to a method for preparing an instant thickening dry food composition that forms a pudding. The instant thickening protein-fortified pudding powder contains an amount of plant protein, sugar, dehydrated fruit pieces and flavor.

The instant thickened pudding type dessert was formed according to typical industry processing techniques using the step-by-step process below. Table 3 is the list of ingredient ranges in percentage (%).

TABLE 3
Instant Thickening Protein-Fortified Pudding Powder

| Ingredient | % Usage Range |
|---|---|
| Sugar | 40 to 60 |
| Soy protein | 30 to 50 |
| Dehydrated Fruit pieces | 3 to 8 |
| Cocoa | 3 to 5 |
| Natural Flavor | 4 to 6 |
| Mineral | 0.5 to 1.5 |
| Vitamin Premix | 0.5 to 1.5 |

The ingredients were combined and processed according to the following steps to produce the finished instant thickened pudding.

All dry ingredients were mixed in a Hobart N-50 mixer using a paddle attachment on speed setting #1 for 5 to 10 minutes. The powder (50-75 g) was then packaged into individual single serve packets.

For the preparation of the instant thickened pudding, 100 mL to 150 mL of cold water was added to an 8 oz/242 ml disposable Solo™ bowl with lid. One single serve packet (50-75 g) was added to the cold water and the lid was placed onto the container. The bowl was then shaken vigorously by hand for 20 to 30 seconds allowing the instant thickening pudding to hydrate and thicken (set).

The result is an instant thickened pudding type dessert containing a combination of plant protein, sugar, dehydrated fruit pieces and flavor with a pudding-like consistency.

### Example 4: Preparation of a Protein-Fortified Soup with SUPRO® Isolated Soy Protein, Fiber, and Dehydrated Vegetable Pieces

The following example relates to a method for preparing an instant thickening dry food composition that forms a soup. The instant thickening protein-fortified soup powder contains an amount of plant protein, dehydrated vegetable pieces and fiber.

The instant thickened soup was formed according to typical industry processing techniques using the step-by-step process below. Table 4 is the list of ingredient ranges in percentage (%).

TABLE 4
Instant Thickening Protein-Fortified Soup Powder

| Ingredient | % Usage Range |
|---|---|
| Soy protein | 30 to 50 |
| Seasoning blend with dehydrated vegetable pieces | 20 to 40 |
| Fiber | 15 to 25 |
| Sugar | 4 to 10 |
| Fat powder | 4 to 10 |
| Salt | 0.5 to 1.5 |
| Mineral | 0.5 to 1.5 |

The ingredients were combined and processed according to the following steps to produce the finished instant thickening protein-fortified soup powder.

All dry ingredients were mixed in a Hobart N-50 mixer using a paddle attachment on speed setting #1 for 5 to 10 minutes. The instant thickening protein-fortified soup powder (50-75 g) was then packaged into individual single serve packets.

For the preparation of the instant thickened protein-fortified soup, 100 mL to 150 mL of cold water was added to an 8 oz/242 ml disposable Solo™ soup bowl with lid. One single serve packet (50-75 g) was added to the cold water and the lid was placed onto the container. The bowl was then shaken vigorously by hand for 20 to 30 seconds allowing the instant thickening protein-fortified soup to hydrate and thicken (set).

The result is an instant thickened protein-fortified soup containing a combination of plant protein, dehydrated vegetable pieces and fiber with a soup-like consistency.

### Example 5: Preparation of a Ready to Eat Frosting with SUPRO® Isolated Soy Protein

The following example relates to a method for preparing an instant thickening dry food composition that forms a frosting. The powder contains an amount of plant protein, flavor and sugar.

The instant thickened protein-fortified frosting was formed according to typical industry processing techniques using the step-by-step process below. Table 5 is the list of ingredient ranges in percentage (%).

TABLE 5
Instant Thickening Protein-Fortified Frosting Powder

| Ingredient | % Usage Range |
|---|---|
| Sugar | 40 to 60 |
| Soy protein | 30 to 50 |
| Fat powder | 15 to 30 |
| Cocoa powder | 5 to 10 |
| Natural Flavor | 3 to 6 |
| Mineral | 0.5 to 1.5 |
| Sucralose | 0.1 to 0.3 |

The ingredients were combined and processed according to the following steps to produce the finished instant thickened protein-fortified frosting.

All dry ingredients were mixed in a Hobart N-50 mixer using a paddle attachment on speed setting #1 for 5 to 10 minutes. The instant thickening protein-fortified frosting powder (50-75 g) was then packaged into individual single serve packets.

For the preparation of the instant thickened protein-fortified frosting, 100 mL to 150 mL of cold water was added to an 8 oz/242 ml disposable Solo™ bowl with lid. One single serve packet (50-75 g) was added to the cold water and the lid was placed onto the container. The bowl was then shaken vigorously by hand for 20 to 30 seconds allowing the instant thickening protein-fortified frosting powder to hydrate and thicken (set).

The result is an instant thickened protein-fortified frosting containing a combination of plant protein, flavor and sugar with a frosting like consistency.

### Example 6: Preparation of an Instant Thickening Peanut Spread with SUPRO® Isolated Soy Protein, Fiber, and Peanuts

The following example relates to a method for preparing an instant thickening powder that forms a peanut spread. The instant thickening peanut spread powder contains an amount of plant protein, flavor, and powder derived from ground peanuts.

The instant thickening peanut spread was formed according to typical industry processing techniques using the step-by-step process below. Table 6 is the list of ingredient ranges in percentage (%).

TABLE 6
Instant Thickening Peanut Spread Powder

| Ingredient | % Usage Range |
|---|---|
| Soy protein | 30 to 50 |
| Powdered peanut butter | 30 to 40 |
| Fat powder | 15 to 25 |
| Sugar | 8 to 15 |
| Mineral | 0.5 to 1.5 |

The ingredients were combined and processed according to the following steps to produce the finished instant thickening peanut spread.

All dry ingredients were mixed in a Hobart N-50 mixer using a paddle attachment on speed setting #1 for 5 to 10 minutes. The instant thickening peanut spread powder (50-75 g) was then packaged into individual single serve packets.

For the preparation of the finished instant thickening peanut spread, 80 mL to 120 mL of cold water was added to an 8 oz/242 ml disposable Solo™ bowl with lid. One single serve packet (50-75 g) was added to the cold water and the lid was placed onto the container. The bowl was then shaken vigorously by hand for 20 to 30 seconds allowing the instant thickening peanut spread powder to hydrate and thicken (set).

The result is an instant thickened peanut spread containing a combination of plant protein, peanuts and fat with smooth peanut butter like consistency.

### Example 7: Preparation of an Instant Thickening Protein-Fortified Cheese Spread with SUPRO® Isolated Soy Protein, Fiber, and Cheese Flavor

The following example relates to a method for preparing an instant thickening powder that forms a cheese spread. The instant thickening protein-fortified cheese spread powder contains an amount of plant protein, flavor, fat and fiber.

The instant thickening protein-fortified cheese spread was formed according to typical industry processing techniques using the step-by-step process below. Table 7 is the list of ingredient ranges in percentage (%).

TABLE 7
Instant Thickening Protein-Fortified Cheese Spread Powder

| Ingredients | % Usage Range |
|---|---|
| Soy protein | 40 to 60 |
| Fiber | 20 to 40 |
| Cheese flavor | 10 to 15 |
| Fat powder | 10 to 20 |
| Salt | 0.8 to 1.5 |
| Mineral | 0.5 to 1.5 |

The ingredients were combined and processed according to the following steps to produce the finished instant thickening protein-fortified cheese spread.

All dry ingredients were mixed in a Hobart N-50 mixer using a paddle attachment on speed #1 for 5 to 10 minutes. The instant thickening protein-fortified cheese spread powder was then packaged into individual single serve packets of 40 g to 60 g.

For the preparation of the finished instant thickening protein-fortified cheese spread, 100 mL to 150 mL of cold water was added to an 8 oz/242 ml disposable Solo™ bowl with lid. One single serve packet was added to the cold water and the lid was placed onto the container. The bowl was then shaken vigorously by hand for 20 to 30 seconds allowing the instant thickening protein-fortified cheese spread powder to hydrate and thicken (set).

The result is an instant thickened protein-fortified cheese spread containing a combination of plant protein, cheese flavor and fiber with cheese dip like consistency.

### Example 8: Preparation of an Instant Thickening Protein-Fortified Salad Dressing with SUPRO® Isolated Soy Protein and Fiber

The following example relates to a method for preparing an instant thickening powder that forms a salad dressing. The instant thickening protein-fortified salad dressing powder contains an amount of plant protein, flavor, and fiber.

The instant thickening protein-fortified salad dressing was formed according to typical industry processing techniques using the step-by-step process below. Table 8 is the list of ingredient ranges in percentage (%).

TABLE 8
Instant Thickening Protein-Fortified Salad Dressing Powder

| Ingredients | % Usage Range |
|---|---|
| Soy protein | 30 to 50 |
| Fiber | 15 to 30 |
| Sugar | 15 to 30 |
| Fat powder | 15 to 25 |
| Honey mustard flavor | 3 to 5 |
| Mineral | 0.5 to 1.5 |

The ingredients were combined and processed according to the following steps to produce the finished instant thickened protein-fortified salad dressing.

All dry ingredients were mixed in a Hobart N-50 mixer using a paddle attachment on speed setting #1 for 5 to 10 minutes. The instant thickening protein-fortified salad dressing powder (50-75 g) was then packaged into individual single serve packets.

For the preparation of the finished instant thickened protein-fortified salad dressing, 100 mL to 150 mL of cold water was added to an 8 oz/242 ml disposable Solo™ paper cup with lid. One single serve packet (50-75 g) was added to the cold water and the lid was placed onto the container. The cup was then shaken vigorously by hand for 20 to 30 seconds allowing the powder to hydrate and thicken (set).

The result is an instant thickened protein-fortified salad dressing containing a combination of plant protein, flavor, and fiber with salad dressing-like consistency.

### Example 9: Viscosity of Liquid Compositions Produced with Varying Concentrations of Soy Protein and Mineral

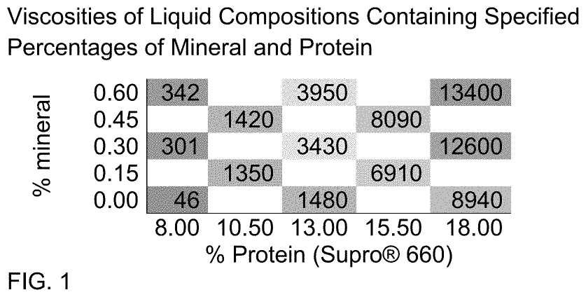

Liquid compositions containing only soy protein at concentrations of 0-18% and/or mineral at concentrations of 0-0.60% in water, by weight of the liquid composition, were produced by mixing soy protein and/or MgO in water. The viscosities of the resulting compositions are shown in FIG. 1. The viscosity of water lacking both soy protein and MgO was approximately 1 centiPoise (cP). The viscosity of compositions containing up to 0.6% MgO in the absence of soy protein was also about 1 cP. The viscosities of compositions containing soy protein in the absence of MgO are shown in FIG. 1. As shown in the chart, compositions having viscosities over 50 cP were produced with as little as 0.15% mineral in combination with soy protein. Surprisingly, although the use of the mineral alone had little effect on the viscosity, the inclusion of a mineral in combination with soy protein significantly increased viscosity over soy protein alone, suggesting a synergistic rheological relationship of soy protein with a mineral.

### Example 10: Viscosity of Liquid Compositions Produced Using Different Minerals

Liquid compositions containing the soy protein of Example 9 and various different minerals were produced to test the impact of the mineral choice on composition viscosity. The results are shown in Table 9.

TABLE 9
Viscosity based on mineral

| | | Viscosity (in cP) after 10 |
|---|---|---|
| | Concentration | minute set time, when mixed |
| Mineral | (%) | with soy protein isolate |
| Calcium chloride | 0.5 | 2 |
| Magnesium chloride | 2.5 | 5 |
| Magnesium oxide | 0.13 | 400 |
| Calcium carbonate | 0.5 | 170 |
| Calcium citrate | 2.75 | 15 |
| Calcium phosphate | 1.5 | 150 |
| Calcium hydroxide | 0.25 | 300 |

### Example 11: Viscosity of Liquid Compositions Produced Using Different Forms of Soy Protein

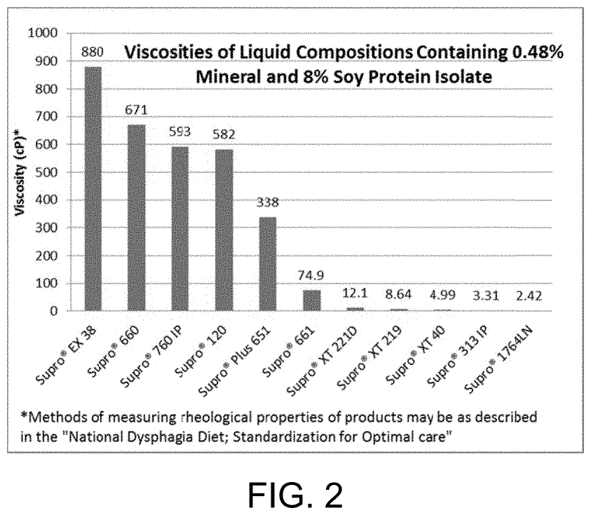

Liquid compositions containing the mineral of Example 9 at 0.48% mineral and 8% of various commercial soy proteins were produced to test the impact of the soy protein choice on composition viscosity. The results are shown in FIG. 2.

### Example 12: Viscosity of Liquid Compositions Produced Using Different Sources of Protein

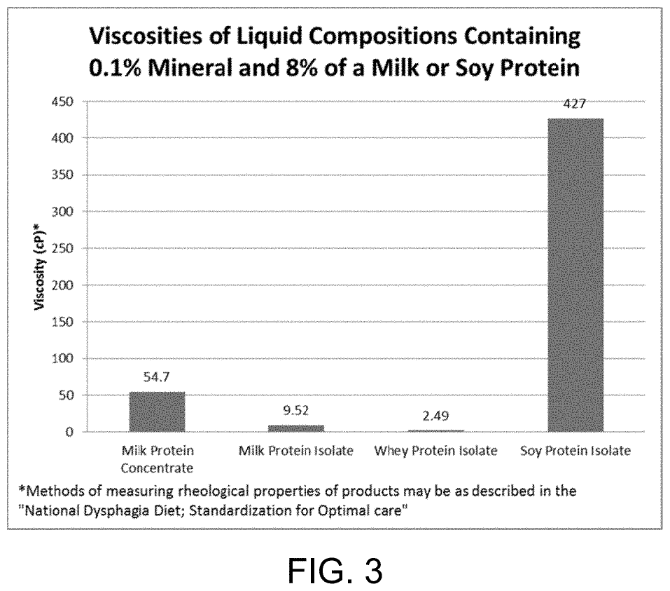

Liquid compositions containing the mineral of example 9 at 0.1% mineral and 8% of various commercial milk or soy proteins were produced to test the impact of the protein choice on composition viscosity. The results are shown in FIG. 3. As shown in FIG. 3, addition of soy protein provided a significant increase in the viscosity over even the most effective milk protein source (427 cP soy protein isolate versus 54.7 cP milk protein concentrate).

One skilled in the art would readily appreciate that the methods, compositions, and products described herein are representative of exemplary embodiments, and not intended as limitations on the scope of the invention. It will be readily apparent to one skilled in the art that varying substitutions and modifications may be made to the present disclosure disclosed herein without departing from the scope and spirit of the invention.

All patents and publications mentioned in the specification are indicative of the levels of those skilled in the art to which the present disclosure pertains. All patents and publications are herein incorporated by reference to the same extent as if each individual publication was specifically and individually indicated as incorporated by reference.

The present disclosure illustratively described herein suitably may be practiced in the absence of any element or elements, limitation or limitations that are not specifically disclosed herein. Thus, for example, in each instance herein any of the terms “comprising,” “consisting essentially of,” and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the present disclosure claimed. Thus, it should be understood that although the present disclosure has been specifically disclosed by preferred embodiments and optional features, modification and variation of the concepts herein disclosed may be resorted to by those skilled in the art, and that such modifications and variations are considered to be within the scope of this invention as defined by the appended claims.

1. An instant thickening dry food composition comprising:
(a) soy protein in an amount ranging from 3% to 99% by weight of the instant thickening dry food composition;
(b) a mineral;
(c) a total hydrocolloid content of not more than 1% by weight of the instant thickening dry food composition; and
wherein the instant thickening dry food composition forms an instant thickened food product when added to a liquid.

2. The instant thickening dry food composition of claim 1, wherein the instant thickened food product is characterized by viscosities of greater than 50 centipoise (cP) as measured by the National Dysphagia Diet guidelines.

3. The instant thickening dry food composition of claim 2, wherein the instant thickened food product comprises at least 8% of the instant thickening dry food composition per weight of the instant thickened food product.

4. The instant thickening dry food composition of claim 1, wherein the mineral comprises a metal cation with a valency of at least 2.

5. The instant thickening dry food composition of claim 4, wherein the metal cation with a valency of at least 2 is magnesium or calcium.

6. The instant thickening dry food composition of claim 1, wherein the total hydrocolloid content represents the total amount of pectin, agar, galactomannans (including fenugreek gum, guar gum, locust bean gum, and tara gum), xanthan gum, cellulose gums (including carboxymethyl cellulose, hydroxypropyl cellulose, hydroxypropyl methylcellulose, microcrystalline cellulose, and methylcellulose), gum Arabic, gellan gum, alginates, alginic acid, arabinoxylans, curdlan gum, cassia gum, konjac gum, carrageenans, gelatins, and starch present in the instant thickening dry food composition.

7. The instant thickening dry food composition of claim 1, further comprising a fiber selected from the group consisting of soluble and/or insoluble fibers of the forms β-glucans, hemicelluloses, lignins, fructans, polyuronide, raffinose, xylose, polydextrose, and lactulose derived from plant and animal sources, and combinations thereof.

8. The instant thickening dry food composition of claim 1, wherein the soy protein is in the form of an isolated soy protein material.

9. The instant thickening dry food composition of claim 8, wherein the isolated soy protein material is selected from the group consisting of SUPRO® 120, SUPRO® 1500, SUPRO® 1610, SUPRO® 1611, SUPRO® 500E, SURPO® 660, SUPRO® 690, SUPRO® 760, SUPRO® 770, SUPRO® 783, SUPRO® EX 32, SUPRO® EX 33, SUPRO® EX37, SUPRO® EX38, SUPRO® EX45, and SUPRO® EX 46, identity-preserved versions of the products, and combinations thereof.

10. The instant thickening dry food composition of claim 1, further comprising at least one additional ingredient selected from the group consisting of dry food pieces, protein-containing materials, bulking agents, carbohydrates (including sweetening agents), dairy products/dairy imitation products, antioxidants, stabilizers, emulsifiers, fats, oils, preservatives, flavoring agents, coloring agents, and combinations thereof.

11. The instant thickening dry food composition of claim 1, wherein the instant thickened food product is selected from the group consisting of baby foods, beverages, custards, cheese imitations, icing/frosting, frozen desserts, imitation yogurts, puddings, spreads, dips, soups, sauces, dressings, and combinations thereof.

12. An instant thickened food product comprising:
(a) an instant thickening dry food composition of claim 1; and
(b) a liquid.

13. The instant thickened food product of claim 12, wherein the liquid is selected from the group consisting of water, milk/milk type (dairy and non-dairy types), fruit juices, vegetable juices, tea, coffee, coconut water, and combinations thereof.

14. The instant thickened food product of claim 12, wherein the instant thickened food product is suitable for administration to subjects with dysphagia.

15. A method for preparing an instant thickened food product, the method comprising the steps of:
(a) adding the instant thickening dry food composition of claim 1 to a liquid; and
(b) stirring or shaking the liquid to which the instant thickening dry food composition is added for a sufficient amount of time to hydrate the instant thickening dry food composition;
whereby the instant thickened food product is prepared.

16. The method of claim 15, wherein the liquid is selected from the group consisting of water, milk/milk type (dairy and non-dairy types), fruit juices, vegetable juices, coffee, tea and combinations thereof.

17. The method of claim 15, wherein the instant thickened food product is suitable for administration to subjects with dysphagia.

18. Use of the instant thickening food composition of claim 1 in the preparation of an instant thickened food product.

19. The use of claim 18, wherein the instant thickened food product is suitable for administration to subjects with dysphagia.

20. Use of the instant thickened food product of claim 1 in the treatment of dysphagia.
